(19) United States
(12) Patent Application Publication
(10) Pub. No.: US 2016/0022575 A1
(43) Pub. Date: Jan. 28, 2016

(54) SYSTEM FOR ADMINISTRATION OF CARDIAC PROTECTIVE THERAPY

(71) Applicant: Peter Costantino, New York, NY (US)

(72) Inventor: Peter Costantino, New York, NY (US)

(21) Appl. No.: 14/809,156

(22) Filed: Jul. 24, 2015

Related U.S. Application Data
(60) Provisional application No. 62/029,436, filed on Jul. 26, 2014.

(51) Int. Cl. A61K 9/00; A61J 1/00; B65D 75/52; A61K 31/616

(52) U.S. Cl. CPC A61K 9/0056; A61K 31/616; A61J 1/00; B65D 75/527

(57) ABSTRACT

An dosage member, comprising a planar member comprising a saliva-dissolvable member, a therapeutic agent carried in said planar member, wherein the saliva-dissolvable member is printed or color-coded to indicate potency. A plurality of aspirin containing dissolvable members may each be color-coded to indicate potency, and include a plurality of green dissolvable members and at least one dissolvable member with a color selected from the group consisting of yellow or amber, a red or red like color and a blue or blue like color. Perforations may provided so that higher potency strips may be divided into lower potency dosages.

### CROSS REFERENCE TO RELATED APPLICATIONS

This application claims the benefit of Provisional Patent Application No. 62/029436, filed Jul. 26, 2014, entitled System for Administration of Cardiac Protective Therapy.

### TECHNICAL FIELD

The invention relates to devices and methods for the prevention and/or treatment of myocardial infarction.

### STATEMENT REGARDING FEDERALLY SPONSORED RESEARCH OR DEVELOPMENT

(Not applicable)

### BACKGROUND OF THE INVENTION

The advantages of regular, for example daily, small dosages of aspirin (2-acetoxybenzoic acid) as a therapeutic agent for reducing the risk of myocardial infarction have long been known. Generally, while the mechanism is not completely understood, aspirin does have an anticoagulant effect in the blood, which is even apparent after administration of daily doses of 30 mg of aspirin for as little as one week, which may be seen by decreased thrombin formation in healthy patients. Thrombin (activated factor II) is a serine protease which converts fibrinogen into an insoluble matrix of fibrin. Fibrin, a protein, cross-links with Factor XIII enzyme and combines with platelets, aggregating platelets into a clot, leading to myocardial infarction, with its attendant reduced availability of oxygen to cells in the tissue of the heart and cellular necrosis.

More particularly, if the vessels that supply the heart with blood are narrowed from atherosclerosis, that is the buildup of fatty deposits on the insides of the arteries, a portion of that fatty deposit lining the inside of the blood vessel can burst thereby becoming thransogenic. Normal blood clotting mechanisms may then form a clot at the site of the ruptured atherosclerotic plaque, and then block the artery. This can substantially cut off blood flow to the heart and cause a heart attack, i.e. death of heart muscle tissue (myocardium).

Aspirin therapy reduces the clumping action of platelets and is generally accepted as reducing the risk of myocardial infarction, body inducing slower prothrombin consumption, thrombin formation and prothrombinase formation at the site of microvascular injury. Aspirin also appears to slow Factor XIII activation by thrombin and to decrease the maximum rate of Factor XIII cleavage. It has also been suggested that there may be a positive correlation between total cholesterol or low-density lipoprotein cholesterol in the amount of thrombin generated after aspirin administration.

Accordingly, daily aspirin therapy is often recommended for persons who have had a heart attack or stroke, persons who have a high risk of having a first heart attack, persons who have had a stent placed in a coronary artery, persons who have had coronary bypass surgery, or individuals with chest pain due to coronary artery disease (angina).

It is not generally recommended that individuals, without medical guidance, commence daily aspirin therapy.

Daily aspirin therapy may not be appropriate for individuals with a health condition that could increase a risk of bleeding or other problem such as a bleeding or clotting disorder, and allergy to aspirin, or bleeding due to a stomach ulcer.

Moreover, if an individual on daily aspirin therapy terminates administration, the body may suffer a withdrawal effect that may increase the risk of heart attack. Terminating daily aspirin therapy they can cause a heart attack by triggering a blood clot.

In addition to acting as a preventative therapy, aspirin administration, if executed quickly enough and if in an effective dosage can be effective to arrest platelet formation even after the mechanisms which cause myocardial infarction have begun. The onset of myocardial infarction may be detected relatively early in the process, even by patients with little education respecting warning signs, sometimes several hours before the tipping point in the process has been reached. The prompt administration of preventative doses of aspirin can arrest and, potentially, reverse the thrombotic phase of the process. This allows the individual time to seek medical attention and determine the advisability of other possible preventative therapies of a more permanent nature, such as anticoagulant therapy, thrombolytic therapy, the insertion of a stent within a vessel, reaming out or flattening fatty deposits on the inside of the artery or coronary artery, bypass and so forth, all on an urgent basis. If an individual is at a medical facility, more elaborate and effective condition monitoring tests may be employed.

The seriousness of delays of sometimes only a few minutes or less is not to be underestimated. While a heart attack does not progress instantly from an extremely dangerous to a severely damaging condition in an instant, the same does occur relatively quickly and the speed with which an intervention may be implemented can make the difference between life and serious injury or even death.

### SUMMARY OF THE INVENTION

Such preventative doses of aspirin, to be most likely to be effective, need to be administered as quickly as possible. Standard procedures currently call for intravenous administration of aspirin. However, such intravenous administration may not be possible, due to the unavailability of the drug and appropriate devices, such as a syringe, inavailability of a skilled individual to administer the drug, or other factor. Likewise, while administration of an appropriate dosage of aspirin orally by tablet may be considered, dosaging by tablet introduces other complications. The patient may be unconscious, incapable of properly swallowing tablets, and so forth. Similarly, even after the tablet is administered, it takes a substantial amount of time for the drug to enter the bloodstream via the gastric lining in the stomach and begin to arrest the conditions causing the onset of myocardial infarction. In accordance with the invention, the preferred method of administration in an emerging setting when intravenous access is not possible is via the oral trans-mucosal route. As a result , pills which are the available form of aspirin, must be chewed to be absorbed even somewhat rapidly via the oral mucosa.

Likewise, daily preventative administration of aspirin can be problematic. Individuals often forget to take the medication. When they do remember, they may or may not have the medication with them. Even if the individual does have medication on his person and senses the onset of myocardial infarction, tablet administration may be ineffective to prevent substantial damage.

The present invention proceeds from the inventive recognition that current procedures for dealing with the onset and/or prevention of cardiac events, while effective in a great number of cases, suffers from these important inadequacies.

Administration is, in accordance with the invention, provided by means of dissolvable films which are used in sublingual administration, by positioning the film between the bottom of the tongue and the floor of the oral cavity. The result is prompt absorption directly into the bloodstream, due to the rapid diffusion of the aspirin (which is a relatively small molecule), through the oral mucosa and neighboring tissues into the bloodstream.

Accordingly, the unavailability of the means to administer aspirin intravenously, the inability of the patient to take a tablet due to unconsciousness or other circumstance, the absence of a trained individual and so forth are alleviated. At the same time, the dosing method involves administration materials which may be conveniently stored, for example in a wallet, purse or packet. In accordance with the invention, different dosages in dissolvable film form containing various desired dosages of aspirin may be differentiated by color or shape. The use of dissolvable film also enables easy creation of reduced dosages, for example by folding the film in half or in quarters and tearing along the fold lines.

Additionally, the ingestion of daily aspirin can irritate the gastric (stomach) mucosa and result in gastric bleeding, discomfort, and morbidity. The use of a dissolveable film containing aspirin circumvents the interaction of the gastric mucosa with the aspirin. The oral mucosa is not at risk of bleeding and irritation compared to the gastric mucosa.

In accordance with the invention, an aspirin dosage member, comprises a planar member comprising a thin film of a saliva-dissolvable member. Between 60 mg and 325 mg of aspirin, for example, are carried in the planar member.

In accordance with the invention, the aspirin dosage containing—dissolvable member may be printed or color-coded to indicate potency.

A plurality of aspirin containing saliva dissolvable members may be each color-coded, and include a plurality of green dissolvable members and at least one dissolvable member with a color selected from the group consisting of yellow or amber (for example signifying a relatively low dosage corresponding to a relatively less serious threat), a red or red like color (for example signifying a relatively average dosage corresponding to an average threat) and a blue or blue like color front for example signifying a relatively high dosage and corresponding to a serious threat).

The inventive aspirin containing green members may have a potency of 40 to 120 mg of aspirin, the yellow or amber members may have a potency of 130 to 190 mg of aspirin and the red or red like members may have a potency of 250 to 650 mg of aspirin, and the blue members may have a potency between 485 and 800 mg of aspirin.

The dosage members may be formulated to dissolve in seconds.

The dissolveable members may be contained within a resealable package disposed in the wallet of a user.

Infrastructure may in accordance with the invention be provided for promoting cardiac health, which comprises fixed and mobile medical facilities stocked with the inventive aspirin dosage members for use by emergency personnel at an early stage in the onset of a cardiac incident.

### BRIEF DESCRIPTION OF THE DRAWINGS

The operation of the inventive method and device will become apparent from the following description taken in conjunction with the drawings, in which:

FIG. 1 is a block diagram of a dissolvable strip having a half elliptical shape;

FIG. 2 is a block diagram of an imprinted dissolvable strip indicating the concentration of aspirin contained in that strip;

FIG. 3 is a perspective view of a stack of dissolvable strips contained in a moisture proof envelope incorporating a plastic zipper;

FIG. 4 is a perspective view of a stack of dissolvable strips contained in a moisture proof envelope incorporating a plastic zipper, where different dissolvable strips contain various amounts of aspirin;

FIG. 5 is a perspective view of a stack of dissolvable strips contained in a plastic heat sealed package, where dissolvable strips contain different concentrations of aspirin;

FIG. 6 is a block diagram of an imprinted dissolvable strip with a central perforation, where the print on strip indicates the concentration of aspirin;

FIG. 7 is a block diagram of an imprinted dissolvable strip with horizontal and vertical perforations, where the print on strip indicates the concentration of aspirin;

FIG. 8 is a perspective view of a stack of dissolvable strips with horizontal and vertical perforations contained in a moisture proof envelope incorporating a plastic zipper;

FIG. 9 illustrates a strip incorporating a medication into a plurality of dosage portions and incorporating day of the week indicia; and

FIG. 10 illustrates a collection of strips selected by the consumer for placement into a plastic envelope suitable for storage in a wallet or purse.

### DETAILED DESCRIPTION OF THE PREFERRED EMBODIMENTS

In accordance with the invention, aspirin containing dissolvable strips (membrane or film) are used either for administering a daily dosage of aspirin for the purpose of reducing the likelihood of myocardial infarction or as an intervention to prevent the onset of an incident. While the actual amount of the dosage may vary depending, for example, upon the tolerance of the individual to aspirin or other factors, the typical daily dosage of aspirin is 81 mg in the context of daily dosaging to to reduce the to reduce the likelihood of a myocardial infarction incident. Accordingly, in one embodiment, the inventive film strips may contain 81 mg of aspirin. However, the preferred dose of aspirin in the context of a possible or impending myocardial infarction is 325 mg. Higher and smaller dosages may be made available with dosage amounts in milligrams being printed on the strips, for example, with or without an additional imprint, “aspirin,” to indicate the identity of the medication.

Alternately, a strip perforated into quarters they have the contents of each quarter (or other portion of the strip) imprinted thereon in various amounts on the same strip.

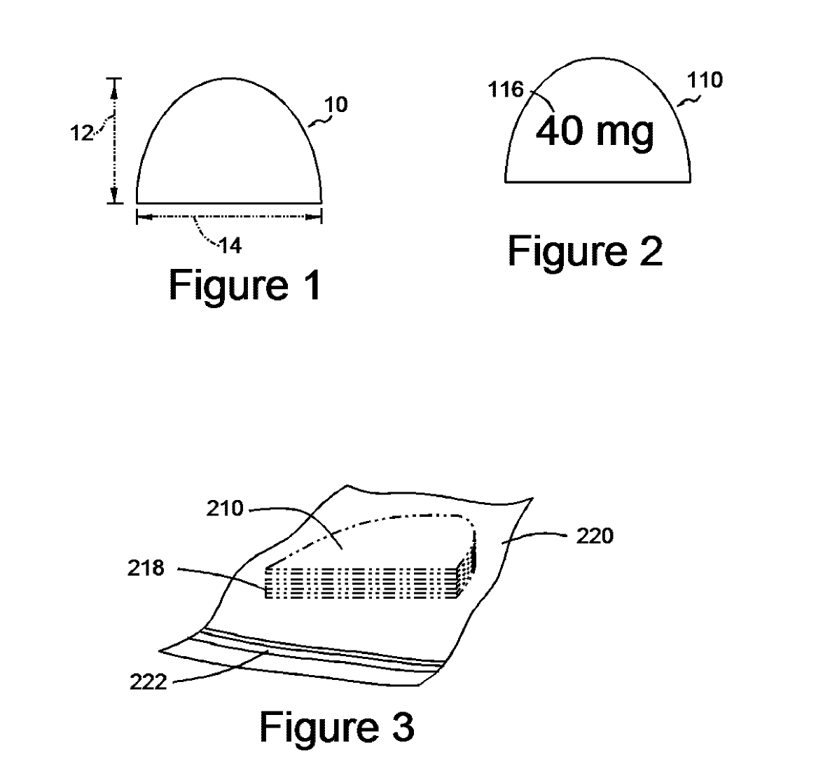

In accordance with the invention, the aspirin containing strips may have a variety of sizes and shapes although circular or oval-like shapes are preferred to maximize size while still being small enough to fit under the tongue of a user. Size can be further increased by using a half elliptical shape, substantially matching the available and easily accessible shape and size of the underside of the tongue, as illustrated in FIG. 1. In accordance with the invention, it is contemplated that such a half elliptical strip 10 would have a height 12 to width is a 14 ratio of approximately three to two, for example, ranging between four to three, and seven to four. A strip 10, suitable for an adult male might have a height of about two centimeters, perhaps ranging in height from about 1.6-2.2 cm.

The above range of ratios of height to width may also be applied to rectangular, oval and circular shaped strips, as well as other strip shapes.

The maximization of size is desirable because it provides for the quicker dissolution of the medication which contacts saliva on a relatively large portion of the surface area of the tongue. This results in contact between strip 10 and a greater amount of saliva and, accordingly, quicker dissolution. Quicker dissolution is further desirable because it allows a quicker absorption of medication into the system. Such quicker dissolution improves outcomes by more quickly bringing more of the medication in contact with the tissues on the underside of the tongue, where it may be directly absorbed into the bloodstream. Preferred sites for administration are: 1) sSublingual (thin mucosa membrane and dense blood vessels, 2) 1Lingual (on the tongue), 3) Bucal mucosa (inside the cheek) which is useful particularly in an unconscious patient.

As alluded to above, strips 10 meant for daily dosage of aspirin may come in daily dosage forms varying between 20 mg and 81 mg (or more going, perhaps, to 325 mg), for example in increments of 20 mg, that is in strengths of 20 mg, 40 mg, 60 mg, 80 mg and so forth up to 320 mg.

A possibly more advantageous alternative embodiment of the invention is illustrated in FIG. 2. In this specification alternative structural elements in embodiments are, to the extent practical, given reference numerals multiples of 100 different from corresponding structure in earlier embodiments. Referring back to FIG. 2, in order to ensure compliance with directions from the doctor, the dissolvable strips 110 may be imprinted with print 116, for example by silkscreening or another similar technique to indicate their strength (for example, extra strength) or dosage (which may be given in milligrams), as illustrated in FIG. 2.

In accordance with the invention, inventive strips are administered, for example, one time each day as may be directed by doctor. In the regular administration of a medication, it is usually desirable for the medication to be administered at about the same time each day, for example, in the morning. However, an individual may forget to take the medicine at the appropriate time. Thus, in accordance with the invention a stack 218 of strips 210 may be stored in a moisture proof envelope, for example a very small zip-lock plastic bag 220 incorporating a plastic zipper 222 of conventional design as illustrated in FIG. 3.

Strips may also be labeled with the days of the week to promote regular administration of nonemergency drugs.

Because the aspirin containing strips are of relatively thin dimension, for example 2.25×1.5×0.1 cm , as in the case of Listerine dissolvable film breath fresheners, they will dissolve quickly for quick absorption. However, even thinner films may be used, provided they are formulated to be sufficiently pliable to avoid breakage. In addition, such relatively thin films may be provided with separators to enhance easy separation of individual doses.

In accordance with a preferred embodiment of the invention, the stack of strips in the plastic envelope may be interleaved with small sheets of paper which may function as paper separators. The paper separators may be either larger than the strips or smaller than the strips, in any direction. Desirably, the paper may be coated with a release agent which promotes separation of the dissolvable strip containing aspirin from the paper separator. Such coating may be, for example, of wax or a polymeric material. Alternatively, the separators may be made wholly of a polymeric material, or of multilayer construction.

In accordance with the invention, it is contemplated that aspirin containing dissolvable strips may be color-coded, for example, being colored green in the case of a normal, for example, 75 mg dosage. Stronger dosages may be color-coded yellow. Still stronger dosages may be color-coded red while the strongest dosages indicating critical situations may be color-coded blue. Color coding gives the inventive aspirin containing dissolved strips easily ascertainable dosage information. The above colors follow an intuitive scale that will be readily comprehended during use.

In accordance with the invention, it is contemplated that such color-coded strips may be made available, for example, in an ambulance, mobile health facility, emergency room, or other medical setting where individuals trained in the meaning and dosages of the various color-coded aspirin-containing strips will use. Because such strips are color-coded, identification of proper dosages will be quicker than otherwise. Likewise, the possibility of error in dosage administration will be greatly reduced. Both of these factors will contribute to a higher likelihood of successful administration and treatment.

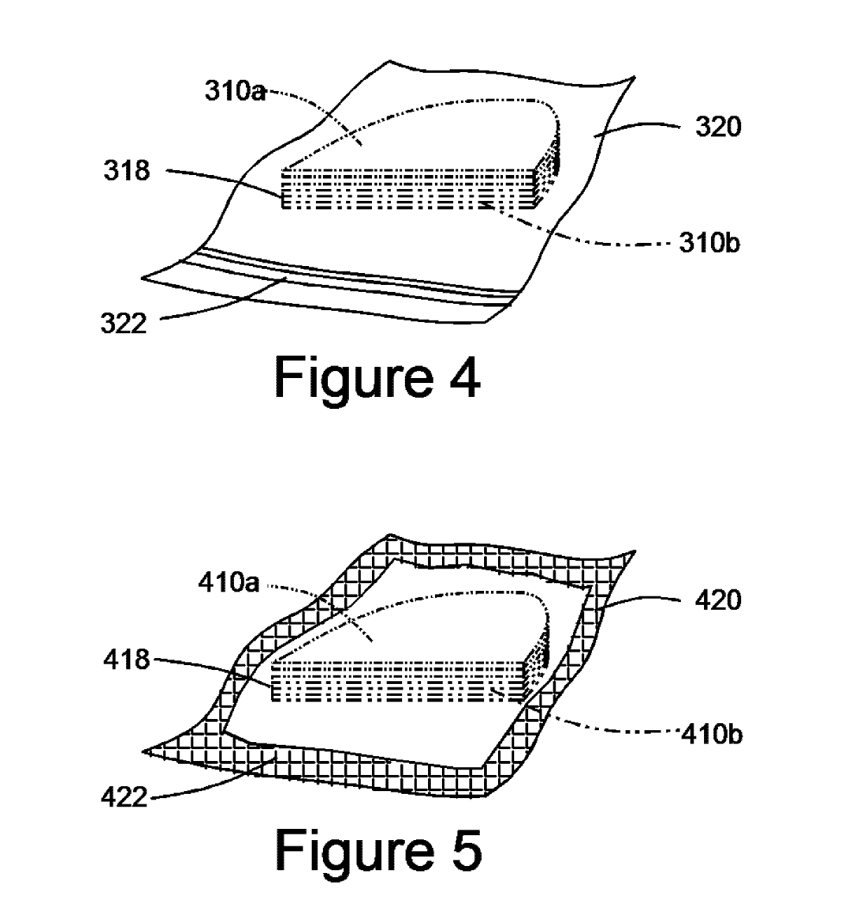

The possibility also exists to make the inventive emergency dosage devices available to individuals. For example, a package such as that illustrated in FIG. 4 containing three red dosages 310a and four yellow dosages 310b may be contained in a single envelope 320. Alternatively, envelopes may carry a single strength of breath strips, and multiple envelopes may be used by individuals.

Because of the relatively thin nature of the aspirin-containing strips, a large number of the same may be contained in a relatively thin zip-lock® type plastic bag and the same may be conveniently stored in a briefcase, bag, or even a wallet or business card holder. This is of particular value, insofar as many individuals requiring regular aspirin administration are of relatively advanced age and may easily forget to take the proper dosage at the appointed time. The ability to store a number of dosages in a wallet, for example, enables an individual to take the proper dosage immediately upon remembering to do the same. Moreover, even if the individual remembers to do the same while in a situation where it is not possible to obtain a glass of water to take a conventional tablet, for example while traveling on a train, and even while driving a car, the needed dosage of aspirin may be easily taken by simply placing the strip under the tongue.

So yet another advantage of such portable packages as illustrated in, for example, FIG. 4, is in the event that the individual begins to get signs of a potential heart attack, the quick availability of the inventive strips encourages prompt action. Moreover, the availability of two, three, or even four different strengths in a single consumer package allows one to match response to the severity of the symptoms experienced.

For example, if an individual feels a sudden great pain in the chest, he can quickly administer a blue very high dosage aspirin containing dissolvable strip.

Likewise, instead of ignoring possible warning signs and waiting to see if they go away, or, worse, attributing them to heartburn, muscle soreness, or other less serious, noncardiac causes an individual would be encouraged to take a yellow or red aspirin containing dissolvable strip. The importance of such easy to take preventative measures is particularly valuable in the case of individuals over the age of 65, as well as persons with heart risk factors, such as obesity, smoking, high cholesterol, high blood pressure, or a family history of cardiac problems.

The ability of the invention to provide a low to no risk alternative to merely waiting, perhaps to the point where serious injury becomes likely, is of particular value insofar as there are numerous signs of impending heart attack which can easily be explained away, including such ordinary things as dizziness, fatigue, irregular heartbeat, nausea, anxiety, shortness of breath, chest discomfort or pain (including pain in areas other than the chest, such as the arms, elbows and shoulders), or persistent coughing and wheezing as a result of fluid accumulation in the lungs due to congestive heart failure.

In accordance with the invention, it is believed that relatively large differentials in the strength of the various color-coded aspirin-containing dissolvable strips is desirable. More particularly, it is contemplated that strengths should be selected so that the use of two strips at one time is discouraged, as dissolution times can be doubled by this practice. It is contemplated that green strips will carry approximately 80 mg of aspirin, although strengths ranging from 40 to 20 mg of aspirin may be employed. Likewise, amber strips might contain about 160 mg of aspirin, while an amber dosage in the range between 130 and 190 mg may be appropriate.

The dosage of red aspirin-containing dissolvable strips may be selected to be 320 mg although dosages in the range of 250 to 650 mg may be employed. Blue aspirin containing dissolvable strips in accordance with the present invention are contemplated to have a dosage of 640 mg, although dosages ranging between 480 and 800 mg, or even higher, may be employed.

In accordance with the invention, green strips are contemplated to be taken daily. In the event that minor symptoms are experienced, a second green strip may be taken at the time of the symptoms. More serious symptoms would involve administration of a yellow strip.

Individuals would be directed in the event of persistence of symptoms, for perhaps 20 minutes after administration of a yellow strip, to take a red strip and take additional red strips if the conditions persist or worsen. Likewise, a maximum dosage would be directed.

Blue strips may be reserved for clear signs of an existing heart attack, for example severe chest pain, and individuals would be directed to take blue strips at intervals of 2-10 minutes, until the maximum number perhaps representing 3000 mg have been consumed. Such blue strips may, optionally, only be carried by ambulence crew members, emergency room personel, urgent care givers, etc.

Directions may also be key to the particular symptoms and the level of severity, with dosages specified in accordance therewith.

Such directions are printed on literature accompanying the inventive aspirin containing dissolvable strips, and in a shorter form on the plastic zip-lock bag containing the strips.

Moreover, such directions inform the individual experiencing the symptoms to promptly and directly seek medical attention by going to an emergency room or other health facility. Such direction further indicates that, for example, high potency aspirin containing dissolvable films are not meant for self treatment or delay of professional treatment but as a means to increase the likelihood of survival long enough to enable professional to address the serious issues created by a potential heart attack.

As noted above, the aspirin-containing dissolvable strips are desirably color-coded. To be most effective, it is anticipated that such color coding shall be of bright shades of green, yellow (or amber), red and blue, so that the same are discernible even under lowlight conditions. Such shades may be, for example, as light as pea green, taxicab yellow, pink and robin's egg blue. In addition, the dissolvable strips are preferably transparent, whereby their color will be seen even when held for viewing with a bright object behind, such as a white table or window open to the sky, or against sunlight.

The inventive method also contemplates separate packaging of higher dosage aspirin containing dissolvable strips. For example, as illustrated in FIG. 5, a plastic package 420 may be sealed hermetically using heat sealing techniques to produce a heat sealed area 422. Because heat sealing is substantially permanent, emergency higher dosage strips, such as yellow strips 410a and red strips 410b may be kept by the individual in a wallet, in the form of heat sealed package 420, together with a second package containing only the lower dosage green strips, such as package 220 illustrated in FIG. 3.

Alternatively to a soft vinyl packet 220, the emergency strips may be carried in a rigid plastic case, for example a case with a snap closure and living hinge.

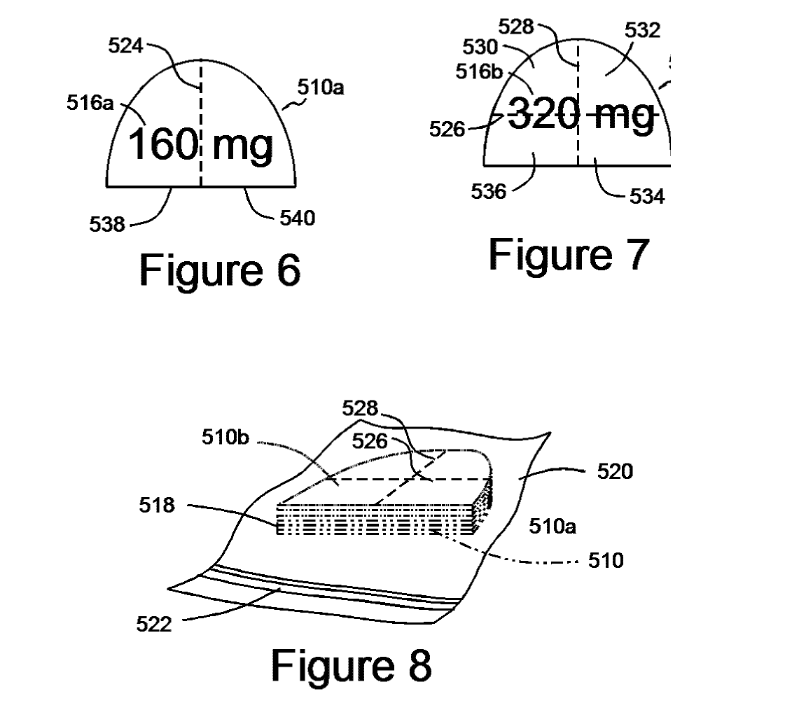

If desired, higher dosage strips may be provided with perforations to allow them to be split into individual standard green strip dosages. For example, as illustrated in FIGS. 6 and 7, 160 mg potency aspirin containing dissolvable strips 510a may be provided with a central perforation 524 allowing them to be folded and easily separated into two equal 80 mg potency dosages (FIG. 6). As shown in FIG. 7, a 320 mg potency red dissolvable strip pay be provided with horizontal perforations 526 and vertical perforations 528. These perforations are optionally placed so that the four quadrants 530, 532, 534 and 536 are of equal area and each contain an 80 mg dosage. Accordingly, each of the halves 538 and 540 of strip 510a, and each of the quarters 530, 532, 534 and 536 of strip 510b each represent the equivalent of 80 mg green strip aspirin dosage of strip 510. Of course, it is understood that these dosages are illustrative and various dosages may be employed.

The advantage of the use of a package 520 (FIG. 8) containing both everyday green dosage strips, and emergency yellow and red strips, of the type illustrated in FIGS. 6-7, is that the emergency strips may be consumed before they become old and are wasted.

The manufacture of dissolvable strips containing medication is well known. For example, the inventive aspirin containing strips may be made by mixing a liquid composition including aspirin in a mixer at various speeds and optionally under pressure for a sufficient period of time to achieve uniformity in the mix. The liquid composition may then be poured onto a polymer sheet, and spread to a uniform thickness. The polymer sheet may be a conveyor belt on which the material travels to and through a drying station where it is allowed to dry. The dried film containing the aspirin may then be further processed, for example by cutting the film into strips. Alternatively, film-forming material, which may be gel or more preferably pullulan based, may be cast by being sprayed into molds and dried using substantially non-moving or circulating or otherwise moving hot air.

Soluble strip compositions typically comprise plasticizers, coloring agents and flavoring agents, plasticizers such as carrageenan, xanthan, locust bean or gum Arabic, solubilizers such as citric acid, tartaric malic acid, modified starches, triacetin and gelatin, flavoring agents such as acesulfame potassium, sucralose, aspartame and stevia, and an emulsifier such as soy lecithin. Strips may also be made to contain multiple active agents.

As alluded to above, strips may also be labeled with the days of the week, thus insuring against missing a dose or taking an extra dose by mistake. Thus a zip lock®-like plastic envelope may contain a plurality of strips, for example seven strips, each labeled with the day of the week. Further in accordance with the invention multiple medications, for example in multiple colors may be included in the envelope. Further in accordance with the invention, a plurality of strips, for example seven, may be included on a single large strip and separated by perforations. Each of the seven strips is labeled with the days of the week. Several such large strips may be included in a plastic envelope, for example large strips containing seven doses of a multivitamin, seven mini doses of aspirin, seven doses of CoQ10, seven doses of vitamin E, and so forth. This would facilitate the daily administration of such environments and supplements with a high degree of reliability.

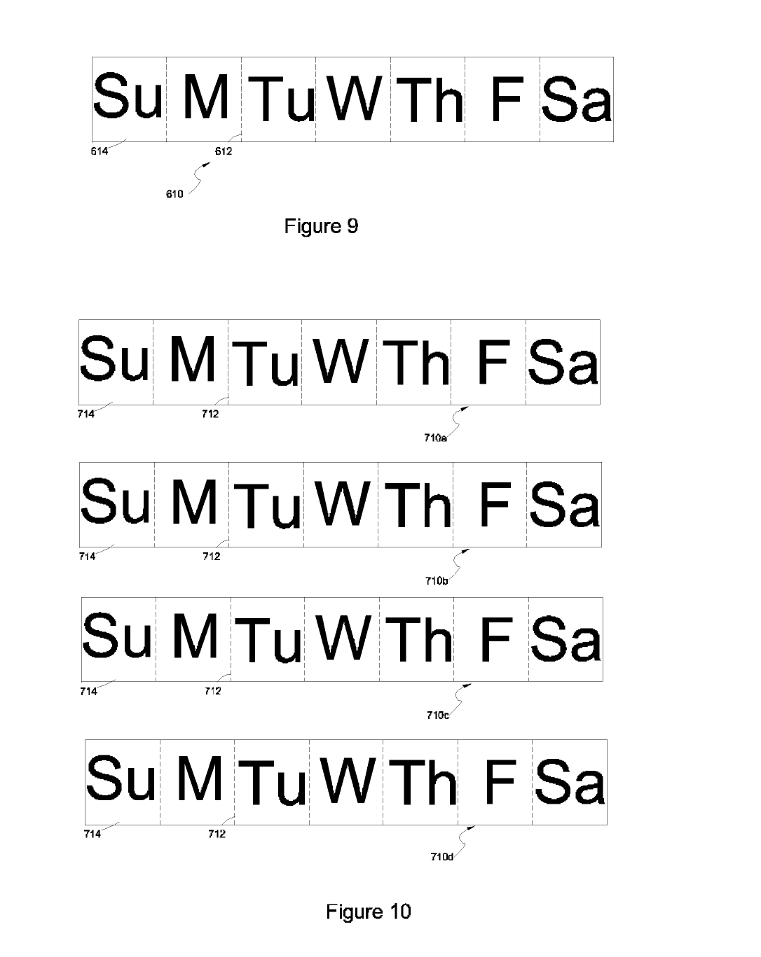

Referring to FIG. 9, a strip 610 incorporating a medication is divided by perforations 612 into a plurality of dosage portions 614 which may be separated into daily dosages by pulling apart at perforations 612. In accordance of the invention strip 610 may comprise a multivitamin supplement.

Referring to FIG. 10, a collection of a plurality of strips 714, 714a, 714b, and 714c each respectively containing a different medication or supplement, such as seven doses of a multivitamin, seven mini doses of aspirin, seven doses of CoQ10, and seven doses of vitamin E may be sold individually, or in packages containing a plurality of one particular medication or supplement and selected by the consumer.

For example, the consumer may buy a package of 30 aspirin strips 714, and also buy a second package of 30 multivitamin strips. The same consumer may also select packages of vitamin E and CoQ10 30 strip packages. That consumer can then take one strip from each of the four packages he has selected and combine them in a vinyl film holder and place them in, for example, a billfold. This would enable convenient, reliable dosaging of these medicines and supplements for a week.

While illustrative embodiments of the invention have been described, it is noted that various modifications will be apparent to those of ordinary skill in the art in view of the above description and drawings. Such modifications are within the scope of the invention which is limited and defined only by the following claims.

What is claimed:

1. An aspirin dosage member, comprising:
(a) a planar member comprising a saliva-dissolvable member;
(b) 60-325 mg of aspirin carried in said planar member.

2. Aspirin dosage as in claim 1, wherein the saliva-dissolvable member is printed or color-coded to indicate potency.

3. A plurality of aspirin containing dissolvable members as in claim 2, wherein saliva dissolvable members are each color-coded, and including a plurality of green dissolvable members and at least one dissolvable member with a color selected from the group consisting of yellow or amber, a red or red like color and a blue or blue like color.

4. A plurality of aspirin containing dissolvable members as in claim 3, wherein said green members have a potency of 40 to 120 mg of aspirin, said yellow or amber members have a potency of 130 to 190 mg of aspirin and said red or red like members have a potency of 250 to 650 mg of aspirin, and set blue members have a potency between 485 and 800 mg of aspirin.

5. An dosage member, comprising:
(a) a planar member comprising a saliva-dissolvable member;
(b) a therapeutic agent carried in said planar member, wherein the saliva-dissolvable member is printed or color-coded to indicate potency.

6. A plurality of dosage members as in claim 5, wherein said therapeutic agent is aspirin and said planar member is formulated to dissolve in seconds.

7. A plurality of dosage members as in claim 6, wherein saliva dissolvable members are each color-coded, and including a plurality of green dissolvable members and at least one dissolvable member with a color selected from the group consisting of yellow or amber, a red or red like color and a blue or blue like color.

8. A plurality of aspirin containing dissolvable members as in claim 7, wherein said green members have a potency of 40 to 120 mg of aspirin, said yellow or amber members have a potency of 130 to 190 mg of aspirin and said red or red like members have a potency of 250 to 650 mg of aspirin, and set blue members have a potency between 485 and 800 mg of aspirin.

9. A plurality of aspirin containing dissolvable members as in claim 8, said dissolvable members being contained within a resealable package disposed in the wallet of a user.

10. A plurality of aspirin containing dissolvable members as in claim 5, wherein said dissolvable members are contained within a resealable package disposed in the wallet of a user, each of said dissolvable members containing between 65 and 95 mg of aspirin.

11. Infrastructure for promoting cardiac health, comprising fixed and mobile medical facilities, said fixed and mobile medical facilities being stocked with aspirin dosage members, comprising:
(a) a planar member comprising a saliva-dissolvable member;
(b) 60-80 mg of aspirin carried in said planar member.

12. Infrastructure for promoting cardiac health wherein the saliva-dissolvable member is printed or color-coded to indicate potency.

13. Infrastructure for promoting cardiac health as in claim 12, wherein saliva dissolvable members are each color-coded, and including a plurality of green dissolvable members and at least one dissolvable member with a color selected from the group consisting of yellow or amber, a red or red like color and a blue or blue like color.

14. Infrastructure for promoting cardiac health as in claim 13, wherein said green members have a potency of 40 to 120 mg of aspirin, said yellow or amber members have a potency of 130 to 190 mg of aspirin and said red or red like members have a potency of 250 to 650 mg of aspirin, and set blue members have a potency between 485 and 800 mg of aspirin.

15. A package containing a plurality of different potency dosage members, comprising:
(a) a plurality of first planar members each comprising a saliva-dissolvable member and having a first quantity of aspirin contained therein;
(b) a plurality of second planar members each comprising a saliva-dissolvable member and having a second quantity of aspirin contained therein; and
(c) a package containing said first and second planar members.

16. A package containing a plurality of different potency dosage members as in claim 15, wherein each of said planar members is provided with perforations allowing said planar member to be divided into lower dosage members.

17. A package containing a plurality of different potency dosage members, comprising:
(a) a first planar member comprising a saliva-dissolvable member and having a first quantity of a vitamin, medication or supplement contained therein, and wherein said first planar member incorporates six perforations positioned to allow said first planar member to be divided into seven individual portions by ripping along said perforations, and further comprising a visible indicia corresponding to the days of the week associated with a respective one of said individual portions of said first planar member;
(b) a second planar member comprising a saliva-dissolvable member and having a second quantity of a vitamin, medication or supplement contained therein, and wherein said second planar member incorporates six perforations positioned to allow said second planar member to be divided into seven individual portions by ripping along said perforations, and further comprising a visible indicia corresponding to the days of the week associated with a respective one of said individual portions of said second planar member; and
(c) a package containing said first and second planar members.

18. An aspirin dosage member is in claim one wherein said planar member is configured as half of a curved object.

19. An aspirin dosage member as an claim 18, wherein the planar member is perforated for division into individual doses of equal strength.
